(19) United States
(12) Patent Application Publication
(10) Pub. No.: US 2025/0325432 A1
(43) Pub. Date: Oct. 23, 2025

(54) MEDICAL DEVICE HOLDING SYSTEM

(71) Applicant: Tejal Megan Jamidar, Daytona Beach, FL (US)

(72) Inventor: Tejal Megan Jamidar, Daytona Beach, FL (US)

(21) Appl. No.: 18/637,669

(22) Filed: Apr. 17, 2024

(51) Int. Cl. A61B 90/00

(52) U.S. Cl. CPC A61G 15/16; A61B 50/20; A61B 2050/21

(57) ABSTRACT

A medical or dental device holding system includes a holder bar with a plurality of device slots. Each of the plurality of device slots includes an inner cavity with an opening and an attachment mechanism located opposite the opening. The attachment mechanism can be a magnet or magnetic device for secure storage and efficient removal. The holding system securely stores medical or dental devices for quick and efficient retrieval.

### BACKGROUND OF THE INVENTION

The present invention relates to medical device holders, and, more particularly, to a magnetic medical device holding system.

During dental procedures, dentists and other personnel need quick and reliable access to tools such as fluid evacuation devices, and air/water syringes. Current devices for providing access include device holders that rely on recessed grooves as a means to secure tools. However, tools can easily be dislodged from recessed grooves by inadvertent contact or improper seating. Once dislodged, a tool must be sterilized or disinfected if it contacts a non-sterile surface, which can add additional time to a procedure.

As can be seen, there is a need for a medical device holding system that can provide quick and efficient access to tools while still providing secure seating when a tool is not in use.

### SUMMARY OF THE INVENTION

In one aspect of the present invention, a medical device holding system is provided. The holding system can include a body with a plurality of device slots. Each device slot can include an opening and a cavity which are adapted to receive a medical device. Each slot can have an attachment mechanism associated therewith, and the attachment mechanism can be positioned opposite of the opening and inside of the body. The attachment mechanism can be a magnet or magnetic retention device.

In another aspect of the present invention, a medical device hanger comprises a holder body with at least one device recess to accommodate a handheld medical device. A magnet is embedded in the holder body at a position corresponding to the at least one device recess.

These and other features, aspects and advantages of the present invention will become better understood with reference to the following drawings, description, and claims.

### BRIEF DESCRIPTION OF THE DRAWINGS

FIG. 1 is a perspective view of a medical device holding system according to an embodiment of the present invention, shown in a use condition;

FIG. 2 is a perspective view of a holder bar thereof;

FIG. 3 is a front elevation view thereof;

FIG. 4 is a top plan view thereof;

FIG. 5 is an exploded view of an embodiment of a holder bar, according to aspects of the present invention;

FIG. 6 is a perspective view of a medical device holding system according to another embodiment of the present invention, shown in a use condition; and

FIG. 7 is an exploded detail view thereof.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is a medical device holder with magnetic retention means.

The inventive medical device holder provides a magnetic retention means for securing medical devices of varying sizes having a ferromagnetic element. Beneficially, the utilization of the magnetic retention means provides a more secure attachment interface, which prevents dislodging by inadvertent contact, and accommodates varying sizes of medical devices without having to modify recessed grooves.

For purposes of illustration, aspects of the inventive device holder are described with respect to dental tools, however it should be appreciated that the device holder is not limited to the dental field. The device holder can be equally applied to fields where instruments, tools, and/or devices are required to be held for quick and secure access, such as the medical field as a non-limiting example.

The medical device holder may be made by any known means. For example, the holder body may be three-dimensionally (3D) printed, injection molded, or milled, but the method of manufacture is not limited thereto. The magnets may be placed inside of the body during or after the body is formed.

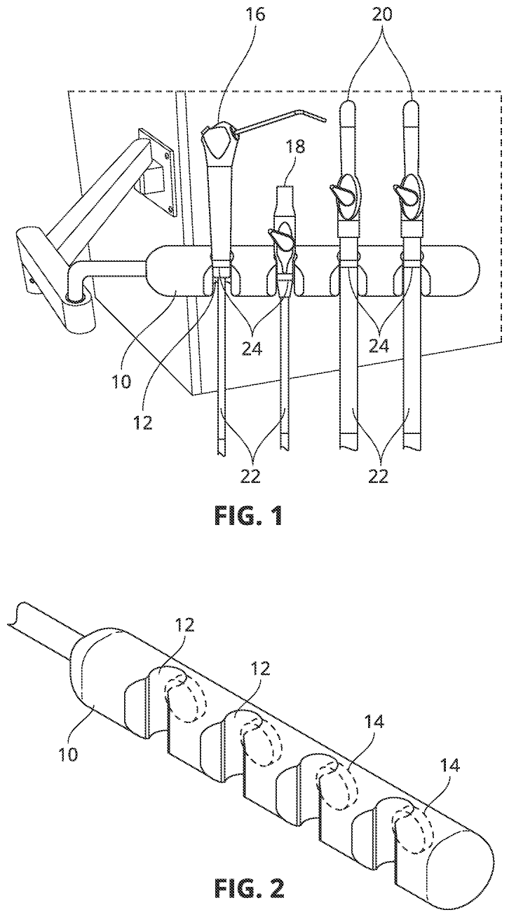

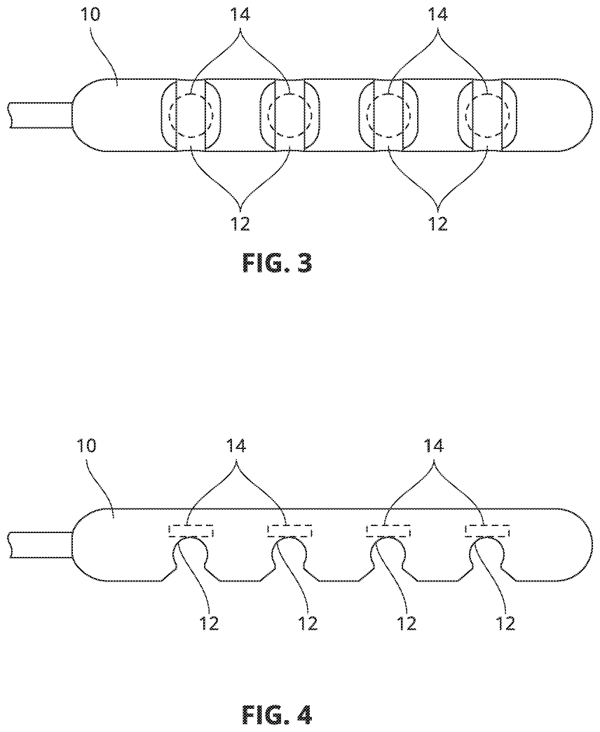

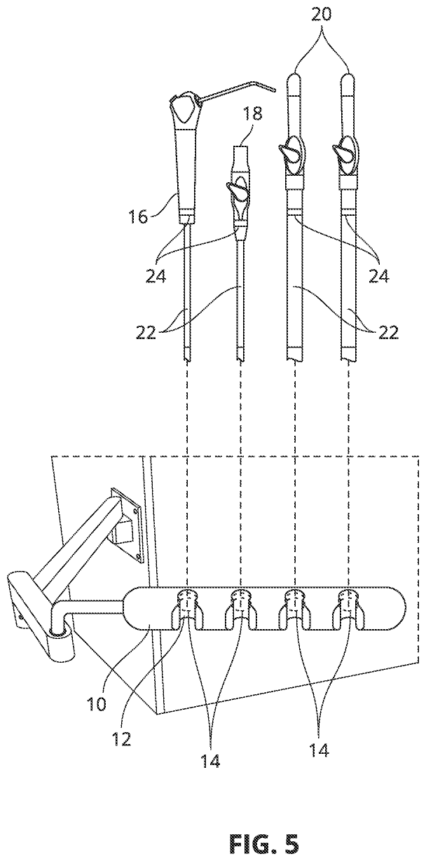

Referring to FIGS. 1-7, FIGS. 1-5 illustrate a first embodiment of a medical device holder, or dental hand tool magnetic mount, according to an embodiment of the present invention. The medical device holder can include a holder bar 10 with a plurality of slots 12 configured to accept a plurality of medical devices, such as dental tools 16, 18 and 20, utilizing a plurality of attachment mechanisms 14. By way of example, dental tools 16, 18, 20 can include high volume fluid evacuation (HVE) devices, low volume evacuation (LVE) devices, also referred to as saliva ejectors, air syringes, and water syringes, but are not limited thereto.

In embodiments, the holder bar 10 can be affixed or otherwise secured to a stable base at a first end, via a telescopic, foldable, or otherwise moveable, arm. Advantageously, fixation by the above mechanism can allow multiple degrees of freedom of movement of holder bar 10, which can provide the ability to place the holder bar 10 in the most advantageous position given the situation. The plurality of slots 12 can be positioned, or spaced, along holder bar 10 to prevent cross-contamination of medical devices 16, 18, and 20. Each of the plurality of slots can include an opening and a cavity adapted to receive and secure medical devices 16, 18, and 20. In embodiments, each cavity can be shaped with an inner diameter, or surface area, capable of accepting any number of medical devices 16, 18, and 20. An opening of each of the plurality of slots 12 can be shaped, or otherwise formed, for quick and efficient removal of medical devices 16, 18, and 20. Specifically, medical devices 16, 18, and 20 with hoses 22 can be accounted for in the shaping of each of the slots 12 openings, to prevent binding and/or catching. It is understood that any shape, and/or size of each of the openings and each of the cavities of the plurality of slots 12 is contemplated so long as the size and shape is adapted to receive, secure, and quickly and efficiently remove medical devices 16, 18, and 20.

Attachment mechanisms 14 can be positioned, or otherwise installed, opposite of each opening of the plurality of slots 12, on an interior aspect of holder bar 10. Attachment mechanisms 14 can provide a secure, contactless, attachment force capable of securing medical devices 16, 18, and 20 within a cavity of a particular slot 12. In embodiments, attachment mechanism 14 can be a magnet that can secure a ferromagnetic material. For a medical device 16, 18, or 20 that includes a ferromagnetic material in its construction, the magnetic attachment mechanism 14 can directly secure the medical device 16, 18, 20 in the slot 12. For a medical device 16, 18, or 20 that does not include ferromagnetic material in its construction, a ferromagnetic ring 24 can be installed on the device to securely mount the device 16, 18, 20 in the slot 12. Advantageously, attachment mechanism 14 in combination with a shape and size of an opening and a cavity of a slot 12 allows for secure attachment of a medical device, while also allowing for quick and efficient retrieval of the medical device.

In embodiments, a user can install a medical device into the system by aligning a hose 22 with an opening of one of the plurality of slots 12, causing the hose to enter a cavity of the slot 12, and lowering the medical device into the cavity such that the attachment mechanism 14 of the slot secures the medical device. In other embodiments, the user can place the medical device 16, 18, 20 through the mouth of the slot 12 and against the attachment mechanism 14.

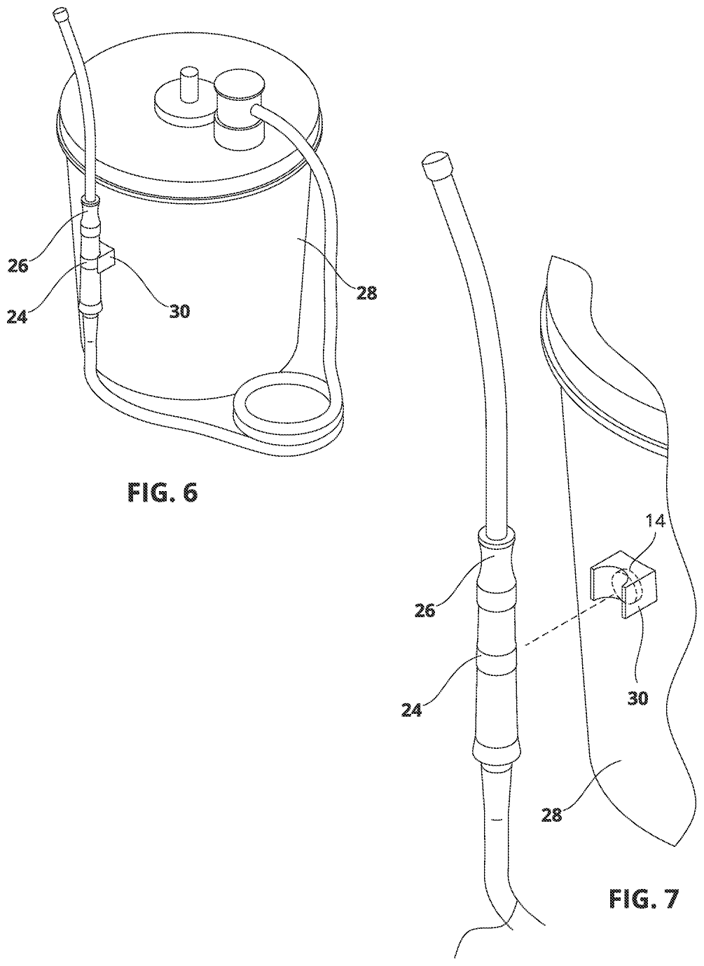

FIGS. 6 and 7 illustrate a medical device mount according to another embodiment of the present invention, comprising a suction system having a collection chamber 28 with a mount 30 on a side wall thereof. A magnet 14 within the mount 30 magnetically couples with a ferromagnetic ring 24 on a suction tool 26, removably retaining it in place.

It should be understood, of course, that the foregoing relates to exemplary embodiments of the invention and that modifications may be made without departing from the spirit and scope of the invention as set forth in the following claims.

What is claimed is:

1. A medical device hanger, comprising a holder body with at least one device recess operative to accommodate a handheld medical device, wherein a magnet is embedded in the holder body at a position corresponding to the at least one device recess.

2. The medical device hanger of claim 1, wherein the holder body is an elongated bar and the handheld medical device is a dental tool selected from the group consisting of: high volume fluid evacuators, low volume evacuators, air syringes, water syringes, and any combination thereof.

3. The medical device hanger of claim 1, wherein the holder body is mounted to a suction canister and the handheld medical device is a suction instrument.

4. The medical device hanger of claim 1, wherein the at least one device recess has a substantially semi-cylindrical wall.

5. The medical device hanger of claim 1, further comprising a ferromagnetic ring adapted to magnetically couple with the magnet and configured to mount on the handheld medical device.

6. The medical device hanger of claim 1, further comprising a repositionable arm and a stable base, wherein the holder body is repositionably fastened to the stable base by way of the repositionable arm.
